(19) United States
(12) Patent Application Publication
(10) Pub. No.: US 2008/0177162 A1
(43) Pub. Date: Jul. 24, 2008

(54) Biosignal measurement apparatus and the method thereof

(75) Inventors: Sang Gon Bae, Seongnam-si (KR); Kun Soo Shin, Seongnam-si (KR); Kun Kook Park, Suwon-si (KR); Soo Kwan Kim, Seongnam-si (KR); Youn Ho Kim, Hwasung-si (KR)

Correspondence Address: STAAS & HALSEY LLP, SUITE 700, 1201 NEW YORK AVENUE, N.W., WASHINGTON, DC, 20005, US

(73) Assignee: SAMSUNG ELECTRONICS CO., LTD., Suwon-si (KR)

(21) Appl. No.: 12/007,685

(22) Filed: Jan. 14, 2008

(30) Foreign Application Priority Data
Jan. 24, 2007 (KR) 10-2007-0007596

(51) Int. Cl. A61B 5/1455

(52) U.S. Cl. 600/323; 600/587

(57) ABSTRACT

A biosignal measurement apparatus including: a headset; a member being detachable from the headset, and being attached onto an ear of a user; a PPG sensor being attached onto the member to detect a PPG signal from the ear of the user; and an acceleration sensor being attached onto the member to detect an acceleration signal due to a motion of the user from the ear of the user is provided.

### CROSS-REFERENCE TO RELATED APPLICATION

This application claims the benefit of Korean Patent Application No. 10-2007-0007596, filed on Jan. 24, 2007, in the Korean Intellectual Property Office, the disclosure of which is incorporated herein by reference.

### BACKGROUND

1. Field

Embodiments relate to a biosignal measurement sensor instrument and a headset instrument having the biosignal measurement sensor instrument and a pendant instrument having the biosignal measurement sensor instrument, and more particularly, to a biosignal measurement sensor instrument which can provide a photoplethysmography (PPG) sensor and an acceleration sensor to a member, detachable from a headset or a pendant, to be adjacent to each other, detect a PPG signal and an acceleration signal from an ear of a user, and eliminate noise in the PPG signal using the acceleration signal when the member contacts with the ear of the user, and thereby can more accurately detect and eliminate dynamic noise included in the PPG signal due to a motion of the user and provide user convenience, and a headset instrument having the biosignal measurement sensor instrument and a pendant instrument having the biosignal measurement sensor instrument

2. Description of the Related Art

As used in the present specification, the term “Ubiquitous” means an information communication environment where a user can be free to access networks at any place without being conscious of the surrounding networks or computers. If ubiquitous is commercialized, anyone can readily use information technology not only at home or in a car, but also even on a mountaintop. Also, the commercialization of Ubiquitous may expand the information technology industry or the scope corresponding thereto by increasing the number of computer users who are connected to networks. Because of its advantage that users can access networks without restriction to time and place, not to mention its portability and convenience, countries worldwide are expanding development and competing in Ubiquitous-related technology now.

Ubiquitous-related technology may be applied to myriad field in human life. In particular, Ubiquitous-HealthCare (hereinafter, U-HealthCare) has recently been in the spotlight as a notable technology area due to the “well-being” boom. U-HealthCare means Ubiquitous technology which enables anyone to readily receive medical services at any time and at any place by installing medical service-related chips or sensors in places of the user's living space. With U-HealthCare, various types of medical attention, such as physical examinations, disease management, emergency care, consultation with a doctor and the like, which currently are only performed in hospitals, may be naturally integrated into our daily lives, thus may be accomplished without going to a hospital.

For example, a diabetic may wear a belt having a blood-sugar management system for blood-sugar management. A blood-sugar sensor attached to the belt may check the blood-sugar of the diabetic upon a specified occasion, and calculate the amount of required insulin corresponding thereto. When the blood-sugar of the diabetic becomes drastically low or high, the belt may provide the blood-sugar information to his/her attending physician using a wireless network, and the attending physician who has received the blood-sugar information may write out an optimal prescription or take the optimal action for the medical emergency.

As an example of U-HealthCare, a portable biosignal measurement device to measure the user's biosignal using an optical sensor is being widely utilized. The user may carry the portable biosignal measurement device at all times and measure various types of biosignals and thereby may prepare for an emergency situation. Accordingly, the portable biosignal measurement device may be regarded as a device capable of showing advantages of U-HealthCare.

The portable biosignal measurement apparatus includes a photoplethysmography (PPG) measurement device. A PPG includes information about a level of peripheral vasoconstriction, and increase and decrease in a cardiac output. Therefore, a physiological status associated with an arterial tube may be understood using the PPG measurement device. Also, the PPG measurement device may be generally utilized as an auxiliary diagnostic device for a particular disease.

Generally, a PPG signal may be measured from a user's finger, earlobe, and the like. Specifically, a detector may detect the user's PPG signal by detecting light, passing through the finger, earlobe, and the like, from a light source. However, when a PPG signal is weak, for example, a PPG signal detected from the earlobe, and the like, a normal PPG signal may not be detected.

When a measurement device measures a PPG signal from a body portion corresponding to a weak signal source, such as the earlobe, and the like, a level of the PPG signal may be less than noise of the measurement device. Specifically, the level of the PPG signal may be less than a system noise level. Therefore, although the weak PPG signal is amplified, the system noise is also amplified and thus a desired PPG signal may not be accurately detected.

As described, when measuring a PPG signal from the earlobe, the most important issue is to eliminate dynamic noise which is caused by a motion of a system. When an apparatus to measure a PPG signal is configured to be portable, the apparatus is generally included in a headset. Specifically, a PPG sensor may be provided on a speaker area of the headset, contacting with the ear of a user, so that the user may readily measure the PPG signal while listening to music using the headset.

However, in this instance, the headset may not closely adhere to the ear of the user at all times and thus a PPG signal may not be accurately measured. Also, significant noise may occur due to the motion of the headset.

Accordingly, there is a need for a portable biosignal measurement device capable of accurately detecting and eliminating dynamic noise, caused by a motion of a user, when measuring a PPG signal from the ear of the user, and thereby providing user convenience.

### SUMMARY

Additional aspects and/or advantages will be set forth in part in the description which follows and, in part, will be apparent from the description, or may be learned by practice of the invention.

An aspect of the present invention provides a biosignal measurement apparatus which can provide a photoplethysmography (PPG) sensor and an acceleration sensor to be adjacent to each other to detect an acceleration signal having greater relation with dynamic noise included in a PPG signal, and eliminate the dynamic noise in the PPG signal using the acceleration signal, and thereby can more accurately measure a PPG signal of a user. In this instance, the PPG sensor detects the PPG signal from (the ear of) the user. Also, the acceleration sensor detects the acceleration signal from (the ear of) the user.

Another aspect of the present invention also provides a biosignal measurement headset device which can provide a PPG sensor and an acceleration sensor to a member, detachable from a headset and attached onto the ear of a user, to be adjacent to each other, and detect an acceleration signal having greater relation with dynamic noise included in a PPG signal and eliminate the dynamic noise in the PPG signal, and thereby can more accurately measure the PPG signal. In this instance, the PPG sensor detects the PPG signal from the ear of the user. Also, the acceleration sensor detects the acceleration signal from the ear of the user.

Another aspect of the present invention also provides a biosignal measurement pendant device which can provide a PPG sensor and an acceleration sensor to a member, detachable from a pendant and attached onto the ear of a user, to be adjacent to each other, and detect an acceleration signal having greater relation with dynamic noise included in a PPG signal and eliminate the dynamic noise in the PPG signal, and thereby can more accurately measure the PPG signal. In this instance, the PPG sensor detects the PPG signal from the ear of the user. Also, the acceleration sensor detects the acceleration signal from the ear of the user.

According to an aspect of the present invention, there is provided a biosignal measurement sensor device including: a member being attached onto an ear of a user; a PPG sensor being attached onto the member to detect a PPG signal from the ear of the user; and an acceleration sensor being attached onto the member to detect an acceleration signal due to a motion of the user from the ear of the user.

According to another aspect of the present invention, there is provided a biosignal measurement headset device including: a headset; a member being detachable from the headset, and being attached onto an ear of a user; a PPG sensor being attached onto the member to detect a PPG signal from the ear of the user; and an acceleration sensor being attached onto the member to detect an acceleration signal due to a motion of the user from the ear of the user.

According to still another aspect of the present invention, there is provided a biosignal measurement pendant device including: a pendant; a member being detachable from the pendant, and being attached onto an ear of a user; a PPG sensor being attached onto the member to detect a PPG signal from the ear of the user; and an acceleration sensor being attached onto the member to detect an acceleration signal due to a motion of the user from the ear of the user.

Additional aspects, features, and/or advantages of the invention will be set forth in part in the description which follows and, in part, will be apparent from the description, or may be learned by practice of the invention.

### BRIEF DESCRIPTION OF THE DRAWINGS

These and/or other aspects, features, and advantages of the invention will become apparent and more readily appreciated from the following description of exemplary embodiments, taken in conjunction with the accompanying drawings of which:

FIG. 1 is a block diagram illustrates a configuration of a biosignal measurement pendant device according to an exemplary embodiment of the present invention;

FIG. 2 illustrates a substantially configured form of a biosignal measurement pendant apparatus according to an exemplary embodiment of the present invention;

FIG. 3 is a block diagram illustrating a configuration of a biosignal measurement headset device according to an exemplary embodiment of the present invention;

FIG. 4 illustrates a substantially configured form of a biosignal measurement headset device according to an exemplary embodiment of the present invention; and

FIG. 5 is a block diagram illustrating a configuration of a biosignal measurement sensor device according to an exemplary embodiment of the present invention.

### DETAILED DESCRIPTION OF THE EMBODIMENTS

Reference will now be made in detail to exemplary embodiments of the present invention, examples of which are illustrated in the accompanying drawings, wherein like reference numerals refer to the like elements throughout. Exemplary embodiments are described below to explain the present invention by referring to the figures.

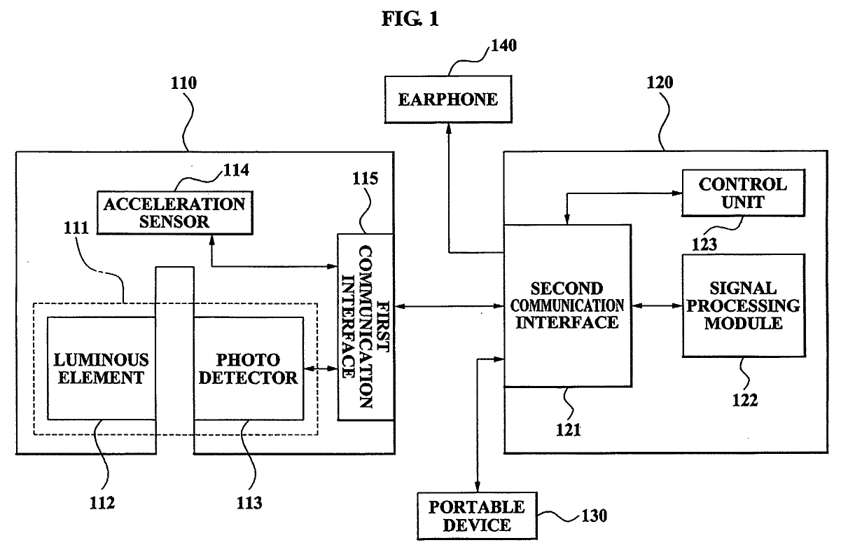

FIG. 1 is a block diagram illustrating a configuration of a biosignal measurement pendant device according to an exemplary embodiment of the present invention.

The biosignal measurement pendant device according to the present exemplary embodiment includes a member 110 and a pendant 120.

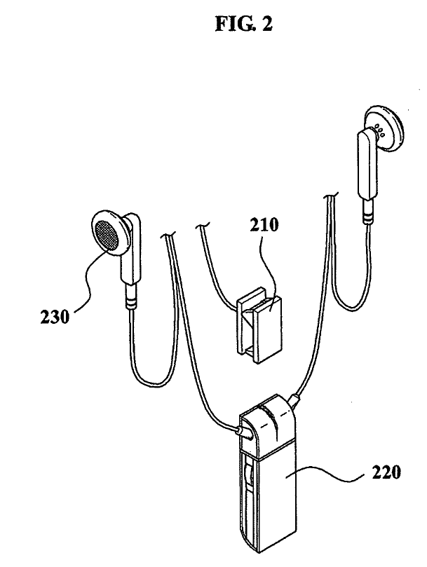

The member 110 may be designed to be detachable from the pendant 120. The form of the member 110 and the pendant 120 according to an exemplary embodiment of the present invention will be described with reference to FIG. 2.

FIG. 2 illustrates a substantially configured form of a biosignal measurement pendant device according to an exemplary embodiment of the present invention.

As shown in FIG. 2, a pendant 220 may be embodied in a form of a necklace. The pendant 220 may connect with a portable device, such as a Motion Picture Experts Group Audio Layer 3 (MP3) player, a mobile communication terminal, a compact disc (CD) player, a portable game device, and the like. Also, the pendant 220 may connect the portable device and an earphone 230. Specifically, when the portable device corresponds to an MP3 player, the pendant 220 may receive sound from the MP3 player and provide a user with the sound using the earphone 230.

In this instance, a member 210 may be detachable from the pendant 220. Specifically, the member 210 may be integrally attached onto the pendant 220. Also, the member 210 may be detached from the pendant 220 by a user and then attached onto an ear of the user. As shown in FIG. 2, the member 210 may be formed in a shape of tongs. Specifically, the user may detach the member 210 from the pendant 220 and attach the member 210 onto the user's ear so that the tongs may be clipped onto the user's ear. The member 210 and the pendant 220 may connect with each other using a wired line or using a local communication network.

Referring again to FIG. 1, the member 110 where a photoplethysmography (PPG) sensor 111, an acceleration sensor 114, and a first communication interface 115 are accommodated in.

The PPG sensor 111 includes a luminous element 112 and a photo detector 113. As described above with reference to FIG. 2, according to an exemplary embodiment of the present invention, the member 110 may be formed in a shape of tongs. In FIG. 1, when the member 110 is formed in the shape of tongs, the luminous element 112 and the photo detector 113 may be provided on inner surfaces of the tongs respectively. Specifically, when the member 110 formed in the shape of tongs is clipped onto the ear of the user, the luminous element 112 and the photo detector 113 may be attached onto both sides of the ear of the user, respectively, and thereby closely contact with the ear of the user.

The luminous element 112 may include a light emitting diode (LED, not shown). Also, in addition to the LED, the luminous element 112 may include any type of material, which can emit light towards the skin of the user to measure a PPG signal.

The photo detector 113 detects light from the ear of the user. Specifically, the photo detector 113 detects the light, which is emitted from the luminous element 112 towards the ear of the user, from the ear of the user.

An acceleration sensor 114 is attached onto the member 110 to detect an acceleration signal due to a motion of the user from the ear of the user. The acceleration sensor 114 may be provided to be adjacent to the PPG sensor 111. Specifically, when the member 110 in the shape of tongs is closely clipped onto the ear of the user, the acceleration sensor 114 is provided in parallel with the PPG sensor 111 contacting with the ear of the user and thereby may measure the acceleration signal from the ear of the user.

As described above, when the PPG sensor 111 and the acceleration sensor 114 are provided to be adjacent to each other, a dynamic noise signal due to the motion of the user may be more accurately detected using the acceleration sensor 114. Specifically, the dynamic noise signal occurs due to the motion of the user and is included in the PPG signal. In this instance, the dynamic noise signal may be more accurately eliminated by using the acceleration signal which is detected using the acceleration sensor 114.

The dynamic noise signal included in the PPG signal is physically different from the acceleration signal. However, when the acceleration signal and the dynamic noise signal are significantly related, the dynamic noise signal may be accurately detected and eliminated in the PPG signal. Accordingly, to accurately detect the dynamic noise signal, a measurement location of the acceleration signal should be set to a location having a greater relation with a measurement location of the PPG signal.

Specifically, in the biosignal measurement pendant device according to the present exemplary embodiment shown in FIG. 1, when the PPG sensor 111 and the acceleration sensor 114 are attached onto the member 110 to be adjacent to each other, it is possible to increase the relation between the acceleration signal and the dynamic noise signal. Specifically, it is possible to accurately detect and eliminate the dynamic noise signal included in the PPG signal by using the acceleration signal.

Also, in FIG. 1, when the member 110 is provided separately from the pendant 120 and an earphone 140, and the member 110 is closely clipped onto the ear of the user to be motionless, it is possible to reduce a noise signal occurrence of a system, which may be caused by motion of the earphone 140 or the pendant 120. Accordingly, it is possible to more accurately detect an acceleration signal having greater relation with the dynamic noise signal included in the PPG signal. It is understood that a shape of the member is not limited to.

The first communication interface 115 is located in the member 110. The first communication interface 115 transmits the detected PPG signal and the acceleration signal to the pendant 120. In this instance, the first communication interface 115 may be configured as a predetermined input/output terminal to make a wired connection with the pendant 120. Also, the first communication interface 115 may be configured as a predetermined local communication module to make a wireless connection with the pendant 120.

A second communication interface 121, a signal processing module 122, and a control unit 123 are accommodated in the pendant 120.

The second communication interface 121 receives the PPG signal and the acceleration signal from the member 110. In this instance, the second communication interface 121 may be configured as a predetermined input/output terminal to make a wired connection with the member 110. Also, the second communication interface 121 may be configured as a predetermined local communication module to make a wireless connection with the member 110.

The signal processing module 122 eliminates the dynamic noise signal included in the PPG signal by using the acceleration signal. As the dynamic noise signal is eliminated, the signal processing module 122 may more accurately measure a PPG signal of the user. Specifically, the signal processing module 122 may eliminate the dynamic noise signal, and also may create various types of biosignal information of the user from the PPG signal in which the dynamic noise signal is eliminated.

The signal processing module 122 may transmit the PPG signal in which the dynamic noise signal is eliminated, to a portable device 130 using the second communication interface 121. In this instance, the second communication interface 121 may be configured as a predetermined input/output terminal to make a wired connection with the portable device 130. Also, the second communication interface 121 may be configured as a predetermined local communication module to make a wireless connection with the portable device 130.

Also, unless the signal processing module 122 eliminates the dynamic noise signal in the PPG signal by using the acceleration signal, the control unit 123 may transmit the PPG signal and the acceleration signal to the portable device 130, which are received from the member 110, using the second communication interface 121. Specifically, instead of the pendant 120, the portable device 130 may eliminate the dynamic noise signal in the PPG signal. Also, the control unit 123 may receive a sound signal from the portable device 130, and output the sound signal using the earphone 140.

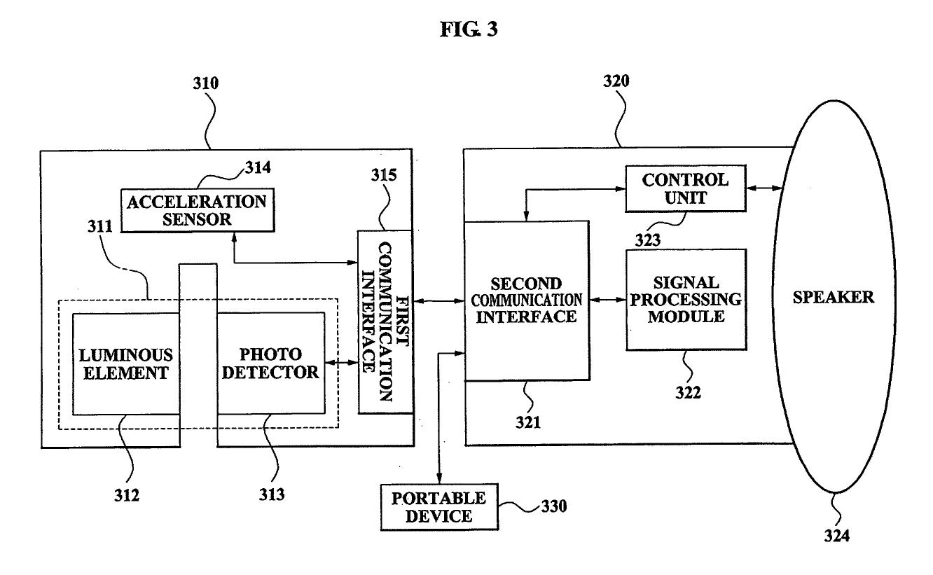

FIG. 3 is a block diagram illustrating a configuration of a biosignal measurement headset device according to an exemplary embodiment of the present invention.

The biosignal measurement headset device according to the present exemplary embodiment includes a member 310 and a headset 320.

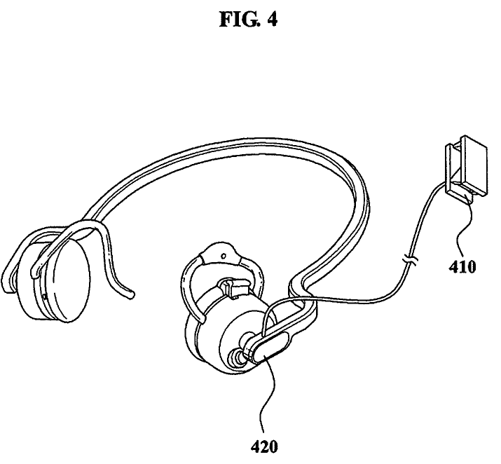

The member 310 may be designed to be detachable from the headset 320. The form of the member 310 and the headset 320 according to an exemplary embodiment of the present invention will be described with reference to FIG. 4.

FIG. 4 illustrates a substantially configured form of a biosignal measurement headset device according to an exemplary embodiment of the present invention.

A headset 420 may connect with a portable device, such as an MP3 player, a mobile communication terminal, a CD player, a portable game device, and the like. A member 410 may be detachable from the headset 420. Specifically, the member 410 may be integrally attached onto the headset 420. Also, the member 410 may be detached from the headset 420 by a user and then attached onto the ear of the user. In this instance, the member 410 may be formed in a shape of tongs as shown in FIG. 4. Specifically, the user may detach the member 410 from the headset 420 and then attach the member 410 in the shape of tongs onto the user's ear so that the tongs may be clipped on the user's ear. The member 410 and the headset 420 may connect with each other using wireless interface, a wired line or a local communication network.

Referring again to FIG. 3, the member 310 includes a PPG sensor 311, an acceleration sensor 314, and a first communication interface 315.

The PPG sensor 311 includes a luminous element 312 and a photo detector 313. As described above with reference to FIG. 4, according to the present exemplary embodiment, the member 310 may be formed in a shape of tongs. In FIG. 3, when the member 310 is formed in the shape of tongs, the luminous element 312 and the photo detector 313 may be provided on inner surfaces of the tongs respectively. Specifically, when the member 310 formed in the shape of tongs is clipped onto the ear of the user, the luminous element 312 and the photo detector 313 may be attached onto both sides of the ear of the user, respectively, and thereby closely contact with the ear of the user.

The luminous element 312 may include an LED (not shown). Also, in addition to the LED, the luminous element 312 may include any type of material, which is widely utilized in the art to emit light towards the skin of the user to measure the PPG signal.

The photo detector 313 detects light from the ear of the user. Specifically, the photo detector 313 detects the light, which is emitted from the luminous element 312 towards the ear of the user, from the ear of the user.

An acceleration sensor 314 is attached onto the member 310 to detect an acceleration signal due to a motion of the user from the ear of the user. The acceleration sensor 314 may be provided to be adjacent to the PPG sensor 311. Specifically, when the member 310 in the shape of tongs is closely clipped onto the ear of the user, the acceleration sensor 314 is provided in parallel with the PPG sensor 311 contacting with the ear of the user and thereby may measure the acceleration signal from the ear of the user.

As described above, when the PPG sensor 311 and the acceleration sensor 314 are provided to be adjacent to each other, a dynamic noise signal due to the motion of the user may be more accurately detected using the acceleration sensor 314. Specifically, the dynamic noise signal occurs due to the motion of the user and is included in the PPG signal. In this instance, the dynamic noise signal may be more accurately eliminated by using the acceleration signal which is detected using the acceleration sensor 314.

The dynamic noise signal included in the PPG signal is physically different from the acceleration signal. However, when the acceleration signal and the dynamic noise signal are significantly related, the dynamic noise signal may be accurately detected and eliminated in the PPG signal. Accordingly, to accurately detect the dynamic noise signal, a measurement location of the acceleration signal should be set to a location having a greater relation with a measurement location of the PPG signal.

Specifically, in the biosignal measurement headset device according to the present exemplary embodiment shown in FIG. 1, when the PPG sensor 311 and the acceleration sensor 314 are attached onto the member 310 in the shape of tongs to be adjacent to each other, it is possible to increase the relation between the acceleration signal and the dynamic noise signal. Specifically, it is possible to accurately detect and eliminate the dynamic noise signal included in the PPG signal by using the acceleration signal.

Also, in FIG. 3, when the member 310, where the PPG sensor 311 and the acceleration sensor 314 are accommodated in, is provided separately from the headset 320, and the member 310 in the shape of tongs is closely clipped onto the ear of the user without to be motionless, it is possible to reduce a noise signal occurrence of a system, which may be caused by motion of the headset 320. Accordingly, it is possible to more accurately detect an acceleration signal having greater relation with the dynamic noise signal included in the PPG signal.

The first communication interface 315 is accommodated in the member 310. The first communication interface 315 transmits the detected PPG signal and the acceleration signal to the headset 320. In this instance, the first communication interface 315 may be configured as a predetermined input/output terminal to make a wired connection with the headset 320. Also, the first communication interface 315 may be configured as a predetermined local communication module to make a wireless connection with the headset 320.

The headset 320 includes a second communication interface 321, a signal processing module 322, and a control unit 323.

The second communication interface 321 receives the PPG signal and the acceleration signal from the member 310. In this instance, the second communication interface 321 may be configured as a predetermined input/output terminal to make a wired connection with the member 310. Also, the second communication interface 321 may be configured as a predetermined local communication module to make a wireless connection with the member 310.

The signal processing module 322 eliminates the dynamic noise signal included in the PPG signal by using the acceleration signal. As the dynamic noise signal is eliminated, the signal processing module 322 may more accurately measure a PPG signal of the user. Specifically, the signal processing module 322 may eliminate the dynamic noise signal, and also may create various types of biosignal information of the user from the PPG signal in which the dynamic noise signal is eliminated.

The signal processing module 322 may transmit the PPG signal in which the dynamic noise signal is eliminated, to a portable device 330 using the second communication interface 321. In this instance, the second communication interface 321 may be configured as a predetermined input/output terminal to make a wired connection with the portable device 330. Also, the second communication interface 321 may be configured as a predetermined local communication module to make a wireless connection with the portable device 330.

Also, unless the signal processing module 322 eliminates the dynamic noise signal in the PPG signal by using the acceleration signal, the control unit 323 may transmit the PPG signal and the acceleration signal, which are received from the member 310, to the portable device 330 using the second communication interface 321. Specifically, instead of the headset 320, the portable device 330 may eliminate the dynamic noise signal in the PPG signal. Also, the control unit 323 may receive a sound signal from the portable device 330, and output the sound signal using a speaker 324.

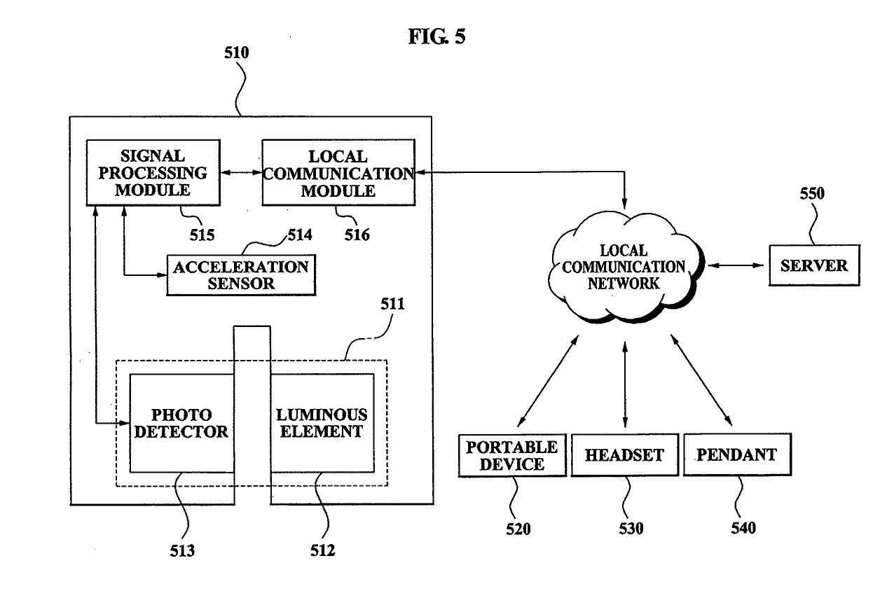

FIG. 5 is a block diagram illustrating a configuration of a biosignal measurement sensor instrument 510 according to an exemplary embodiment of the present invention.

The biosignal measurement sensor instrument 510 according to the present exemplary embodiment may be embodied as an independent configuration from the above-described biosignal measurement headset instrument or pendant instrument. Specifically, the biosignal measurement sensor instrument 510 may be configured to measure a PPG signal and an acceleration signal, eliminate a dynamic noise signal in the PPG signal by using the acceleration signal, and transmit at least one of the PPG signal, the acceleration signal, and the PPG signal in which the dynamic noise signal is eliminated, to an external device.

The biosignal measurement sensor device 510 includes a PPG sensor 511, an acceleration sensor 514, a signal processing module, 515, and a local communication module 516.

The PPG sensor 511 includes a luminous element 512 and a photo detector 513. The biosignal measurement sensor device 510 may be formed in a shape of tongs. When the biosignal measurement sensor device 510 is formed in the shape of tongs, the luminous element 512 and the photo detector 513 may be provided on inner surfaces of the tongs respectively. Specifically, when the biosignal measurement sensor device 510 formed in the shape of tongs is clipped onto the ear of the user, the luminous element 512 and the photo detector 513 may be attached onto both sides of the ear of the user, respectively, and thereby closely contact with the ear of the user.

The luminous element 512 may include an LED. Also, in addition to the LED, the luminous element 512 may include any type of material, which is widely utilized in the art to emit light towards the skin of the user to measure the PPG signal.

The photo detector 513 detects light from the ear of the user. Specifically, the photo detector 513 detects the light, which is emitted from the luminous element 512 towards the ear of the user, from the ear of the user.

An acceleration sensor 514 detects an acceleration signal due to a motion of the user from the ear of the user. The acceleration sensor 514 may be provided to be adjacent to the PPG sensor 511. Specifically, when the biosignal measurement sensor instrument 510 shape of tongs is closely clipped onto the ear of the user, the acceleration sensor 514 is provided in parallel with the PPG sensor 511 contacting with the ear of the user, and thereby may measure the acceleration signal from the ear of the user.

As described above, when the PPG sensor 511 and the acceleration sensor 514 are provided to be adjacent to each other, a dynamic noise signal due to the motion of the user may be more accurately detected using the acceleration sensor 514. Specifically, the dynamic noise signal occurs due to the motion of the user and is included in the PPG signal. In this instance, the dynamic noise signal may be more accurately eliminated by using the acceleration signal which is detected using the acceleration sensor 514.

The dynamic noise signal included in the PPG signal is physically different from the acceleration signal. However, when the acceleration signal and the dynamic noise signal are significantly related, the dynamic noise signal may be accurately detected and eliminated in the PPG signal. Accordingly, to accurately detect the dynamic noise signal, a measurement location of the acceleration signal should be set to a location having a greater relation with a measurement location of the PPG signal.

Specifically, in the biosignal measurement sensor device 510 according to the present exemplary embodiment, when the PPG sensor 511 and the acceleration sensor 514 are attached onto the biosignal measurement sensor instrument 510 in the shape of tongs to be adjacent to each other, it is possible to increase the relation between the acceleration signal and the dynamic noise signal. Specifically, it is possible to accurately detect and eliminate the dynamic noise signal included in the PPG signal by using the acceleration signal.

Also, in FIG. 5, when the biosignal measurement sensor device 510 including the PPG sensor 511 and the acceleration sensor 514 is formed in the shape of tongs, and the biosignal measurement sensor instrument 510 is closely clipped onto the ear of the user without to be motionless, it is possible to reduce a noise signal occurrence of a system, which may be caused by motion of the biosignal measurement sensor device 510. Accordingly, it is possible to more accurately detect an acceleration signal having greater relation with the dynamic noise signal included in the PPG signal.

The signal processing module 515 eliminates the dynamic noise signal included in the PPG signal by using the acceleration signal. As the dynamic noise signal is eliminated, the signal processing module 515 may more accurately measure a PPG signal of the user. Specifically, the signal processing module 515 may eliminate the dynamic noise signal, and also may create various types of biosignal information of the user from the PPG signal in which the dynamic noise signal is eliminated.

The signal processing module 515 may transmit the PPG signal in which the dynamic noise signal is eliminated, to any one of a portable device 520, a headset 530, a pendant 540, and a server 550 using the local communication module 516.

Conversely, the signal processing module 515 may be excluded from the biosignal measurement sensor device 510. In this case, the PPG signal and the acceleration signal may be directly transmitted to any one of the portable device 520, the headset 530, the pendant 540, and the server 550.

The biosignal measurement sensor device 510 may be configured to make a local communication with any one of the portable device 520, the headset 530, the pendant 540, and the server 550, and to be detachable from the portable device 520, the headset 530, the pendant 540, and the server 550.

According to the above-described exemplary embodiments of the present invention, there is provided a biosignal measurement sensor device which can provide a PPG sensor and an acceleration sensor to be adjacent to each other to detect an acceleration signal having greater relation with dynamic noise included in a PPG signal, and eliminate the dynamic noise in the PPG signal using the acceleration signal, and thereby can more accurately measure a PPG signal of a user. In this instance, the PPG sensor detects the PPG signal from the ear of the user. Also, the acceleration sensor detects the acceleration signal from the ear of the user.

Also, according to the above-described exemplary embodiments of the present invention, there is provided a biosignal measurement headset device which can provide a PPG sensor and an acceleration sensor to a member, detachable from a headset and attached onto the ear of a user, to be adjacent to each other, and detect an acceleration signal having greater relation with dynamic noise included in a PPG signal and eliminate the dynamic noise in the PPG signal, and thereby can more accurately measure the PPG signal. In this instance, the PPG sensor detects the PPG signal from the ear of the user. Also, the acceleration sensor detects the acceleration signal from the ear of the user.

Also, according to the above-described exemplary embodiments of the present invention, there is provided a biosignal measurement pendant device which can provide a PPG sensor and an acceleration sensor to a member, detachable from a pendant and attached onto the ear of a user, to be adjacent to each other, and detect an acceleration signal having greater relation with dynamic noise included in a PPG signal and eliminate the dynamic noise in the PPG signal, and thereby can more accurately measure the PPG signal. In this instance, the PPG sensor detects the PPG signal from the ear of the user. Also, the acceleration sensor detects the acceleration signal from the ear of the user.

Although a few exemplary embodiments of the present invention have been shown and described, the present invention is not limited to the described exemplary embodiments. Instead, it would be appreciated by those skilled in the art that changes may be made to these exemplary embodiments without departing from the principles and spirit of the invention, the scope of which is defined by the claims and their equivalents.

What is claimed is:

1. A biosignal measurement apparatus comprising:
a photoplethysmography (PPG) sensor to detect a PPG signal from a user; and
an acceleration sensor to detect an acceleration signal due to a motion of the user.

2. The biosignal measurement sensor instrument of claim 1, wherein biosignal measurement apparatus is formed in a shape of tongs.

3. The biosignal measurement apparatus of claim 1, wherein the PPG sensor comprises:
a luminous element to emit light towards the ear of the user; and
a photo detector to detect the light passing through the ear of the user.

4. The biosignal measurement apparatus of claim 1, further comprising:
a signal processing module to eliminate noise in the PPG signal by using the acceleration signal; and
a local communication module to transmit at least one of the PPG signal in which the noise is eliminated, the PPG signal, and the acceleration signal, to an external device.

5. The biosignal measurement apparatus of claim 1, wherein the biosignal measurement apparatus is to be detachable from any one of a headset, a pendant, and a portable device

6. The biosignal measurement apparatus of claim 1, the biosignal measurement apparatus wherein the luminous element is an LED (Light Emit Diode).

7. A biosignal measurement apparatus, comprising:
a member part to sense a PPG signal from a user and to detect an acceleration signal due to a motion of the user; and
a noise elimination part to eliminate noise in the PPG signal by the acceleration signal.

8. The biosignal measurement of claim 7, wherein the member part is formed in a shape of tongs.

9. The biosignal measurement apparatus of claim 7, wherein the member part comprises:
a PPG sensor to detect a PPG signal from the user;
an acceleration sensor to detect an acceleration signal due to the motion of the user; and
a first communication interface to send the acceleration signal to the noise elimination part.

10. The biosignal measurement apparatus of claim 9, wherein the PPG sensor comprises:
a luminous element to emit light towards the user; and
a photo detector to detect the light passing through the user.

11. The biosignal measurement apparatus of claim 7, wherein the noise elimination part comprises:
a signal processing module to eliminate noise in the PPG signal by using the acceleration signal.

12. The biosignal measurement apparatus of claim 7, wherein the noise elimination part further comprises:
a control unit to transmit the PPG signal and the acceleration signal to a portable device.

13. The biosignal measurement apparatus of claim 12, wherein the control unit to receives a sound signal from the portable device.

14. The biosignal measurement apparatus of claim 12, wherein the portable device has a capability of removing a dynamic noise signal in the PPG signal.

15. The biosignal measurement apparatus of claim 7, wherein the noise elimination part is a form of a headset.

16. The biosignal measurement apparatus of claim 15, wherein the headset and the noise elimination part are attachable each other.

17. The biosignal measurement apparatus of claim 7, wherein the noise elimination part is a form of a pendant.

18. The biosignal measurement apparatus of claim 17, wherein the pendant and the noise elimination part are attachable each other.

19. The biosignal measurement apparatus of claim 10, the biosignal measurement apparatus wherein the luminous element is an LED (Light Emit Diode).

20. A method for measuring a biosignal, comprising:
detecting a PPG signal from a user using a photoplethysmography (PPG) sensor;
detecting an acceleration signal due to a motion of the user; and
eliminating noise in the PPG signal using the acceleration signal.
